(19) United States
(12) Patent Application Publication
(10) Pub. No.: US 2014/0276608 A1
(43) Pub. Date: Sep. 18, 2014

(54) BALLOON SCLEROTHERAPY

(71) Applicant: ABBOTT CARDIOVASCULAR SYSTEMS, INC., Santa Clara, CA (US)

(72) Inventors: Wouter E. Roorda, Palo Alto, CA (US); Travis R. Yribarren, Coarsegold, CA (US)

(73) Assignee: ABBOTT CARDIOVASCULAR SYSTEMS, INC., Santa Clara, CA (US)

(21) Appl. No.: 13/802,017

(22) Filed: Mar. 13, 2013

(51) Int. Cl. A61M 25/10

(52) U.S. Cl. CPC A61M 25/10
USPC 604/509; 604/103.01

(57) ABSTRACT

Disclosed are methods, devices, and systems for treating a body lumen with a sclerothepeutic agent. The method includes the steps of positioning a porous balloon assembly at a site in the lumen to be treated, inflating the assembly to establish a close approximation to the lumen wall, forcing the sclerotherapeutic agent to exit the pores and come in contact with the lumen wall, and to deflate and retrieve the balloon assembly. The methods, devices and systems disclosed herein are especially useful for the treatment of incompetent veins and their contributory branches.

### BACKGROUND OF THE INVENTION

1. The Field of the Invention

The invention generally relates to the field of treating body lumens, in particular incompetent veins, with a sclerotherapeutic agent. Specifically, porous balloon assemblies and treatments using such assemblies for a targeted delivery of the agent to the affected sites are disclosed.

2. Background and Relevant Art

Sclerotherapy is the treatment of tissue with a chemical irritant to induce a controlled injury and subsequent healing reaction. It is typically used to obliterate unwanted tissue.

Sclerotherapy of small, superficial varicose veins is a well-established technique, currently using mostly surfactants as sclerotherapeutic agents, like polidocanol and sodium tetradecyl sulphate dissolved in water. The solutions are injected directly by syringe into the varicose veins, often in the form of foams, because of the greater fill factor of foams compared to regular solutions. Because of the low volume and flow in these small varicose veins, limited amounts of the sclerotherapeutic agents are required.

The underlying cause of many visible and sometimes painful varicose veins and their complications is a failure of the valves in a downstream vein bed, mostly the Great Saphenous Vein (GSV). Failure of these valves leads to incompetence of the GSV, causing retrograde flow, pooling of blood, and failure of smaller upstream veins. Surgical removal or percutaneous occlusion of the GSV is often an effective cure for more superficial varicose veins and their complications.

Percutaneous thermal ablation of incompetent GSVs is a common procedure performed in the U.S., Europe and many other countries. The treatment can have a high clinical success rate and is based on heating the tissue of the venous wall to temperatures in excess of 200° F. In the case of radiofrequency ablation, the probe directly heats the venous wall, while in the case of laser ablation, the laser probe actually boils the blood, causing the steam to ablate the vessel wall.

With this procedure, patients may not only need anesthesia, but the surrounding tissue may need to be protected from burn damage as well. Both of these objectives can be achieved by the use of tumescent anesthesia, in which large amounts of a dilute anesthetic solution are infused under pressure around the vein. Unfortunately, the very act of infusing the solution under pressure can be painful and can cause significant patient discomfort and/or necessitate the use of conscious sedation and/or peri-procedural opioids.

In addition, the probes used in thermal ablation oftentimes may not be capable of being directed into small tributary veins and perforator veins. A lack of occlusion of these veins may contribute to later recanalization of the main saphenous vein. Therefore, these veins are commonly removed in a subsequent procedure by stab avulsion, in which a small incision is made in the skin, and the veins extracted with a small hook and ligated.

Efforts to use sclerotherapy for the treatment of incompetent saphenous veins have been undertaken since at least the 1970s. However, because of the size of these veins, large amounts of sclerotherapeutic agent may be required, and, in the case of the use of a foam, large amounts of gas may be introduced into the central circulation.

The treatment has been shown to be potentially effective, but may come at the cost of (transient) neurological side effects in up to 3% of patients, and possible other side effects due to the introductions of significant quantities of surfactant into the bloodstream. Consequently, sclerotherapy of the GSV is not currently approved by the FDA, and the use of sodium tetradecyl sulphate is specifically contraindicated for this application.

### BRIEF SUMMARY OF THE INVENTION

Disclosed are methods, devices and systems for the treatment of body lumens, in particular incompetent veins and their contributory side branches, with a sclerotherapeutic agent.

One embodiment of the present invention includes a method for tumescent anesthesia-free treatment of a body lumen having a lumen wall, which can eliminate the use of tumescent anesthesia, for instance, in sclerotherapeutic procedures. The method can involve positioning an inflatable balloon assembly at a location in the body lumen and forcing a sclerotherapeutic agent to exit the one or more pores of the inflatable balloon assembly and to contact the lumen wall. In addition, the method can involve retaining the sclerotherapeutic agent between the lumen wall and the outer wall of the inflatable balloon assembly when the inflatable balloon assembly is in the inflated configuration.

Another embodiment also can include a method for tumescent anesthesia-free treatment of a body lumen having a lumen wall. Such method can involve unfolding and expanding the multiple porous balloon chambers of an inflatable balloon assembly into an unfolded configuration in a manner that outer walls of the multiple porous balloon chambers abut the lumen wall. Still further, the method can include forcing a sclerotherapeutic agent through one or more pores of the multiple porous balloon chambers to contact the lumen wall. The method also can include retaining the sclerotherapeutic agent between the lumen wall and the outer walls of the multiple porous balloon chambers when the multiple porous balloon chambers are in the unfolded configuration.

Yet another embodiment includes a device for administering a beneficial agent into a body lumen. The device can include a balloon assembly having a compliant balloon chamber with a chamber wall and at least one pore in the chamber wall, the at least one pore being located at a reinforcement in the chamber wall.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. For better understanding, the like elements have been designated by like reference numbers throughout the various accompanying figures. Understanding that these drawings depict only typical embodiments of the invention and are not therefore to be considered to be limiting of its scope, the invention will be described and explained with additional specificity and detail through the use of the accompanying drawings in which:

FIG. 1A illustrates a side view of an uninflated porous balloon assembly in a vein;

FIG. 1B illustrates a side view of an inflated porous balloon in a vein;

FIG. 1C illustrates a side view of an inflated porous balloon in a vein, indicating a space for delivery of a sclerotherapeutic agent.

FIG. 2 illustrates a side view of an exemplary embodiment of the invention;

FIG. 3A illustrates a cross-sectional view of a folded non-compliant balloon;

FIG. 3B illustrates a cross-sectional view of an inflated non-compliant balloon;

FIG. 4A illustrates a cross-sectional view of an uninflated compliant balloon;

FIG. 4B illustrates a cross-sectional view of an inflated compliant balloon;

FIG. 5A illustrates a cross-sectional view of an inflated double-chamber balloon with coaxial chambers;

FIG. 5B illustrates a side view of a double-chamber balloon with a spiral chamber;

FIG. 5C illustrates a side view of a multi-chamber balloon with parallel chambers;

FIG. 6A illustrates an enlarged cross-sectional view of a compliant chamber wall with pores located in reinforcing protrusions in an uninflated state; and

FIG. 6B illustrates an enlarged cross-sectional view of a compliant chamber wall with pores located in reinforcing protrusions in an inflated state.

### DETAILED DESCRIPTION OF THE INVENTION

### Glossary

The terms distal end and distal direction refer to an orientation away from the operator.

The terms proximal end and proximal direction refer to an orientation towards the operator.

Fluids: Flowable materials, including liquids, liquid mixtures, emulsions, suspensions, gases and foams.

Inflation material: Fluid injected into balloon assembly to achieve inflation.

Sclerotherapy: Treatment of a biological tissue with a chemical irritant, often performed to obliterate unwanted tissue or organs.

Sclerotherapeutic agent: Fluid with active ingredient suitable to perform sclerotherapy. Such fluids also may be used as an inflation material.

Incompetent vein: Vein in which valves have lost functionality and may be incapable of preventing retrograde flow of blood.

Varicose vein: Vein that has become dilated and/or tortuous.

Tributary Vein: A smaller vein emptying into a larger vein

Perforator Vein: A vein directly connecting a vein in a deep venous bed with a vein in a superficial venous bed.

Disclosed are treatment methods for body lumens, in particular incompetent veins, with a sclerotherapeutic agent, and porous intravascular balloon systems for use in such treatments. Embodiments of the present invention may provide improved treatments of incompetent and varicose veins. Specifically, the improvements may include lower cost, improved safety and efficacy and improved patient comfort.

Porous intravascular balloons have been studied for the treatment of vascular wall tissue at least since the early 1990s. Most of these early studies were directed at preventing restenosis of coronary arteries after angioplasty and stenting.

Despite significant efforts, the approach was by and large unsuccessful, and was abandoned in favor of drug-eluting stents.

In contradistinction to these earlier studies, it has now been found that in the treatment of incompetent veins the use of porous intravascular balloons can be highly effective, as was demonstrated in an in-vivo study in a large animal model described in more detail further below.

The invention disclosed here includes a porous balloon that may be introduced into the vasculature or other luminal organ by means of a catheter, and may be inflated in the lumen of an incompetent vein, like the Great Saphenous Vein or any other vein or body lumen in need of treatment.

While the treatment method and devices described below are not limited to treatment of veins, treatment of incompetent veins is a major objective of the present invention, and many of the examples and embodiments below will refer to treatment of incompetent veins.

As illustrated in FIG. 1A, a porous balloon assembly 200 in a folded or collapsed, uninflated state may be positioned inside a vein 100 to be treated.

In FIG. 1B the balloon assembly 200 is inflated to abut the vessel wall 110.

Referring to FIG. 1C, a sclerotherapeutic agent (not shown) may be delivered through the pores 210 of the balloon assembly 200 into the space 120 between the inflated balloon assembly 200 and the wall 110 of the vein 100. In this manner the amount of sclerotherapeutic agent delivered to the systemic circulation may be greatly reduced compared to the amount that would be required by straight infusion of the agent into an unobstructed vein with active blood flow.

At the same time the sclerotherapeutic agent may be contained between the inflated balloon assembly 200 and the vessel wall 110 by keeping the balloon assembly 200 inflated for a predetermined amount of time, thereby greatly extending its residence time and further reducing the amount of sclerotherapeutic agent needed. At the end of the procedure, the balloon assembly 200 may be deflated and retrieved from the vein 100, and the access site closed.

An additional advantage may be that administration in this manner may avoid the need to formulate the sclerotherapeutic agent into a foam, reducing the amount of gas introduced into the systemic circulation.

The combined effect of the reductions in usage of gas and sclerotherapeutic agent described above may greatly reduce, or even eliminate, systemic side effects of the treatment, while the ability to maintain a high concentration of sclerotherapeutic agent in the space between the balloon and the vessel wall may improve the efficacy of the treatment.

Also, treatment with a porous intravascular balloon delivering a sclerotherapeutic agent may improve patient comfort by avoiding the need for the potentially painful use of tumescent anesthesia, which may otherwise be required for other treatments of large incompetent and varicose veins, such as treatments using high temperature ablation.

Finally, the cost of the procedure may be reduced compared to thermal ablation by a lower cost of the device, and the absence of the need for tumescent anesthesia, periprocedural opioids and/or conscious sedation of the patient.

As shown in FIG. 2, the devices of the present invention may include a delivery assembly 300 including a catheter shaft 310, a hub assembly 400 on the proximal end of the delivery assembly 300 and a balloon assembly 200 on the distal end of the delivery assembly 300, as well as one or more radio-opaque or echogenic markers 500.

The balloon assembly may contain one or more chambers 220 for inflating the balloon assembly and for delivering the sclerotherapeutic agent to the location to be treated. At least one of the chambers may contain a number of pores 210 for delivery of the sclerotherapeutic agent. While in some embodiments a single pore may be sufficient for the function of the device, a plurality of pores may be preferred in most embodiments.

The delivery assembly 300 may connect the hub assembly 400 to the balloon assembly 200, may provide the means to position the balloon assembly 200 at the location to be treated while maintaining extracorporeal access to the hub 400, and may contain one or more lumens 320 connecting the access port(s) 420 on the hub assembly 400 to the chamber(s) 220 of the balloon assembly 200.

Additionally, the delivery assembly 300 may contain a guidewire lumen 340.

The hub assembly 400 may include a guidewire port 410 to accommodate the use of a guidewire (not shown) and multiple access ports 420 to accommodate such functions as the introduction of one or more fluids (not Shown), further described below, into the balloon assembly 200.

In order to facilitate fluoroscopic or ultrasound detection and guidance of the device, radio-opaque and/or echogenic markers 500 may be incorporated in the design.

The device may be introduced into the vessel through an introducer sheath. The sheath may be equipped with a rotating hemostatic valve to reduce blood loss during the procedure. A guidewire may be used to guide the device to the location to be treated.

Turning now to the balloon assembly.

Balloon materials may have different compliances. Non-compliant and semi-compliant balloons may be constructed from rigid to semi-rigid materials, and are typically collapsed into a folded state as shown in cross-section in FIG. 3A. The balloon chamber 220 can be folded around the guidewire lumen 340, and is shown expanded in FIG. 3B. Compliant balloons may be constructed from somewhat elastic to highly elastic materials, and may be collapsed into a folded state or into an elastically deflated state as shown in FIG. 4A, to be elastically expanded into its inflated state in FIG. 4B.

Non-compliant and semi-compliant balloons may be defined by a nominal pressure, which is the pressure at which the balloon is inflated to a fully unfolded configuration, and has reached a defined, nominal diameter. For instance a “6 mm balloon” may have a 6 mm diameter at a nominal pressure of 10 Atmosphere.

In the context of this disclosure, compliant balloons may expand freely with increased pressure to many times their uninflated diameter. They do not necessarily have a defined nominal diameter. They may be collapsed into an elastically deflated state rather than in a folded state, and therefore may not have a fully unfolded state.

A comprehensive definition of the terms compliant, semi-compliant and non-compliant is provided in U.S. Pat. No. 5,556,383, issued to Wang et al. and entitled “Block Copolymer Elastomer Catheter Balloons,” the contents of which are incorporated herein by reference.

While the behavior and properties of non-compliant balloons are distinctly different from those of fully compliant balloons, the limits described above are for convenience of description only, and do not denote any distinct sharp transition points from one set of properties to the next.

Additionally, more than one material may be used for the balloon assemblies within the scope of the present invention.

Inflation of the balloons assemblies may be achieved by introducing one or more inflation materials into the balloon chambers. Technically, a wide range of fluids may serve as suitable inflation materials, including pure liquid substances, liquid mixtures, solutions, emulsions, suspensions, foams and gases or gas mixtures. In clinical practice water and saline, if desired in heparinized form, may be the most common inflation materials. Additionally, the sclerotherapeutic agent may be used as an inflation material, especially in single chamber balloon assemblies.

The examples below describe embodiments of non-compliant and compliant balloons. It should be understood that many embodiments, including those of semi-compliant balloons, exist that exhibit properties that fall in between those described for the embodiments below.

Turning now to non-compliant balloons.

Non-compliant balloons may be collapsed into a folded state along a longitudinal axis as shown in FIG. 3A to facilitate delivery into the vessel.

Introduction of the inflation materials into the balloons may result in unfolding as the major deployment mechanism of these balloons, rather than in elastic expansion, which may be the case in compliant balloons.

By definition, non-compliant balloons exhibit only a small amount of diametrical growth in response to increased pressure after they reach a fully unfolded condition.

As a consequence, pores present in a non-compliant balloon material may exhibit a similarly small diametrical growth in response to increased pressure. Therefore, the flow into and out of these pores may be predominantly controlled by the diameter and length of the pore, the pressure on the balloon and the viscosity of the formulation to be delivered, and only to minor extent by the elasticity of the porous material and pore growth with pressure.

$$F = \frac{ r 4 Δ p}{8 η L} \qquad Formula 1$$

may be applicable,
where
F=Flow
η=viscosity coefficient
r=pore diameter
L=pore length
Δp=pressure differential

Since these balloons are less likely to adapt to vessel diameter, physicians may be able to accommodate for the different diameters of blood vessels encountered in clinical practice by choosing from a range of unfolded balloon diameters provided by the suppliers of the device. In clinical practice, a range of diameters of 5 mm to 30 mm may be desirable, but diameters outside this range are possible under the scope of the invention. Similarly, balloon lengths from 5 cm to 30 cm may be a clinically desirable range, but other lengths are possible under the scope of the invention.

The requirements for the materials of construction can include properties like biocompatibility, sterilizability, physical and chemical stability, strength and flexibility.

For non-compliant balloons, a high elasticity modulus and a high tensile strength can be desirable in order to construct thin-walled devices that may withstand high inflation pressures. As an example, a frequently used material is Nylon 12, with an elasticity modulus of about 2 Gigapascal and a tensile strength of about 50 Megapascal.

Other suitable materials include, but are not limited to other polyamides, poly-urethanes, poly-esters, poly-ethers, poly-olefines, and poly-(meth) acrylates.

Turning now to compliant balloons.

In the case of highly compliant balloons, the uninflated state may not be a folded configuration, but rather an elastically deflated one, as illustrated in FIG. 4A. Increases in pressure may translate directly into elastic stretching of the balloon wall material and into increases in balloon diameter.

This may have important consequences for the pores in these balloons. With increasing balloon diameter the wall material of the balloon may get thinner, reducing the length of the pore, while at the same time the pore diameter may increase.

$$F = \frac{ r 4 Δ p}{8 η L} \qquad Formula 1$$

may still be applicable,

But in this scenario, the radius r and the length L may become variables, which may need to be taken into account when controlling the flow of the sclerotherapeutic agent from the balloon. Also, longitudinal stretching of the balloon may cause the pores to be farther apart in an inflated state than in an uninflated state.

An advantage of this type of balloon is that one size of balloon may be used for a range of vein sizes.

With balloons like this it may be advantageous to use therapeutic agents of different viscosities for different vessel sizes. For example, when treating larger vessels, a larger balloon diameter and/or a larger pore diameter may be required, in which case a higher viscosity of the sclerotherapeutic agent may be used to reduce the flow rate F.

Therefore, whereas with non- or low-compliance balloons adjustments for the vein size may be preferably made by selecting from a range of balloon sizes, with high compliance balloons these adjustments may be preferably made by selecting from a range of viscosities of the sclerotherapeutic agent.

It should be understood that these adjustments options maybe preferred for the aforementioned balloon types, but that technically all variables in the formula may be available to adjust the flow from any type of balloon.

The requirements for the materials of construction include properties like biocompatibility, sterilizability, physical and chemical stability, strength and flexibility.

For compliant balloons a low elasticity modulus may be a requirement, typically less than 10 Megapascal. Typical materials of construction include latex, silicone and soft poly-urethanes.

Turning now to the pores.

As for the pores, an inherently porous material like ePTFE may be selected, or pores may be created in a non-porous material.

Various methods are available for creating pores in a non-porous balloon material. For instance, laser-drilling of pores and mechanical or laser-cutting of slits may be used to create pores of controlled sizes.

As one of the possible alternatives, a leachable pore former may be incorporated into the balloon material at the time of manufacture of the material. At any desired later stage, the leachable material may be extracted from the balloon material, leaving pores or open, interconnected pathways present through the balloon material.

The pores can have any number of suitable shapes and sizes. For example, the pores may have a circular, oval, rectangular or other cross-section, or they may be irregular in shape as may be the case after the use of a leachable pore former. Longitudinal slots or cuts, made by laser or mechanical cutting may be advantageous in that they may open up in response to increased pressure, even in non-compliant materials, and may create an opportunity to delay significant release of the sclerotherapeutic agent until the balloon has been deployed.

A wide range of pore sizes and pore numbers are feasible. For instance, for balloons constructed from a material like ePTFE, the average pore size may be well under 1 micron. Balloons like this may exhibit a slow oozing of fluid from an extremely large number of pores in response to pressure increases.

At the other extreme, balloon designs with a single pore are in principle feasible as well. The efficacy of such balloon designs may depend on an adequate distribution of the sclerotherapeutic agent around the balloon after exiting the pore. In practice, a distribution of pores around the balloon may facilitate the distribution of the sclerotherapeutic agent and may therefore be preferred.

The desired size of the pores may be determined from Formula 1 above, combined with the desired flow rate and number of pores. If desired, the viscosity of the sclerotherapeutic agent may be adjusted to accommodate a wider range of pore sizes for any balloon type.

Turning now to the delivery assembly.

Single or multiple lumen catheter shafts may be combined with any of the balloon designs described above.

For instance, a catheter shaft with a single lumen 320 may be coupled with a single chamber balloon assembly 200 as illustrated in FIG. 1a-c. The balloon assembly may be inflated and/or unfolded by injecting the sclerotherapeutic agent. In this case, premature release of the sclerotherapeutic agent may be minimized by selecting a combination of pore diameter, pore length (which may be equal to chamber wall thickness) and viscosity of the solution, that requires a substantial pressure in order to release appreciable amounts of sclerotherapeutic agent

In the case of multi-chamber balloons it may be advantageous to use multi-lumen catheter shafts to enable independent operation of the chambers of the balloon.

For instance, in the case of a double chamber balloon connected to a dual lumen catheter shaft it may be advantageous to inflate an inflation chamber in the balloon to its fully unfolded or inflated configuration, allowing the balloon to be positioned optimally versus the vessel wall. Subsequently a sclerotherapeutic agent may be injected into a porous delivery chamber, allowing the agent to exit the pores with the balloon in an optimal position. Several embodiments will be discussed below.

In addition to the lumens described above, a guidewire lumen may be present in the shaft to accommodate the use of a guidewire. As such, the guidewire lumen may not provide access to any of the chambers of the balloon. Fluids may be introduced through the guidewire lumen, but these fluids may have access to the lumen of a blood vessel and may typically contain heparin or other anti-coagulants to prevent clotting of blood in the guidewire lumen.

The size, number and configuration of the lumens in the shaft may be determined based on such factors as the design of the balloon assembly, the desire for the use of a guidewire, the viscosity of the fluid or fluids to be used, the acceptable diameter and flexibility of the shaft.

Turning now to the hub assembly.

The hub assembly may contain the access ports for a guidewire and for connecting the catheter shaft lumen or lumens to fluid delivery devices such as syringes or pressure-regulated inflation devices. An access port for a guidewire may be advantageously located coaxially with a corresponding guidewire lumen to minimize guidewire friction during positioning of the device.

When multiple shaft lumens are present, it may be advantageous to connect individual lumens to a unique access port, in case independent introduction of fluids into the lumens is desired, but this is not required. For instance, if mixing of two or more liquids just before administration is desired, multiple access ports may be connected to a single lumen.

In such cases it may be advantageous to incorporate or connect a static mixer segment to the hub. Static mixers are well known in the art, and typically consist of a hollow cylindrical body with a number of blades or baffles which separate fluid streams into turbulent flow pattern causing efficient mixing without the use of moving parts.

The access ports may be fitted with a variety of valves to control blood loss or to control inflation fluids, but this is not required. In clinical practice, such valves and controls are often present on the devices that are connected to the access ports on the hubs.

Turning now to the markers.

The delivery assembly may also include one or radio opaque markers or echogenic markers to assist in positioning the assembly at desired locations.

Turning now to the sclerotherapeutic agent.

A number of different types of sclerotherapeutic agents have been used in the past for the treatment of small varicose veins. These include surfactant solutions, concentrated salt solutions and mixtures of water and ethanol. Currently, only two sclerotherapeutic compounds are approved by the FDA for the treatment of small varicose veins, sodium tetradecyl sulphate and polidocanol. Neither one is approved for use in the large veins like the Great Saphenous Vein, and sodium tetradecyl sulphate is specifically contraindicated for this use.

During treatment, aqueous mixtures of these compounds are injected into the varicose veins, causing deep endothelial damage and occlusion of the vessel. Similar mixtures can be used for treatment of the larger varicose veins, including the saphenous veins, as described herein. Mixtures of non-prescription compounds, like an ethanol-water mixture, may be suitable as well.

If increased viscosities are desired to control the flow rate from the pores in response to pressure increases, a suitable viscosifier may be added to the mixture. Suitable viscosifiers can be found in the FDA list of inactive ingredients for parenteral use, and include compounds like poly-ethylene glycol, sodium carboxy methyl cellulose and poly-vinyl pyrrolidone.

Turning now to the controls for administering the sclerotherapeutic agent

For example, a syringe, coupled to one of the ports on the hub assembly for administering sclerotherapeutic agent. Dosage control may be achieved by calculating the difference between the amount of sclerotherapeutic agent injected into the device and the amount re-aspirated before retrieval.

As another example, if pressure control is considered important, a pressure-regulated device, like those used for inflating angioplasty balloons may be employed.

In devices with multi-chamber balloon assemblies, combinations of a syringe and a pressure-regulated device may be possible.

### Clinical Use of the Invention

The method of use of the invention may be based on commonly used procedures for percutaneous vascular intervention.

The most commonly treated vein may be a segment of the saphenous vein in the upper leg.

The patient may be placed in a prone position, and the access site prepared in accordance with standard procedures.

Access to the vein may be achieved by a modified Seldinger procedure and an introducer sheath positioned in the vein.

The device may be introduced through the sheath and positioned at the site to be treated. The use of a guidewire may be optional, at the discretion of the operator.

The device may be inflated, and the sclerotherapeutic agent may be caused to exit the pore or pores, enter the venous lumen and contact the vessel wall.

The device may be maintained in position for a determined period of time, after which the balloon may be deflated and retrieved.

Finally, the access site may be closed according to standard procedure.

In clinical practice, an additional advantage of the present invention may be the high steerability and deliverability of balloons made according to contemporary design principles.

Many incompetent veins, like incompetent GSVs, have contributory venous branches like tributary veins and perforator veins. Leaving these contributory branches untreated often contributes to late recanalization of the main vein, and therefore treatment of these contributory veins is typically considered a medical necessity.

In many instances, thermal ablation of the main vein is the accepted standard of care, but many thermal probes are not able to be guided into these contributory branches. Therefore, additional treatments like injection sclerotherapy or stab avulsions are used to occlude these branches.

Many embodiments of the present inventions may be to be delivered over a guidewire, and are therefore capable of being steered into the contributory branches. In this manner the entire treatment of the incompetent vein and its contributory branches may be completed in a single session.

### Exemplary Embodiments

Under the scope of the invention, balloons may be designed having many different shapes, including straight bodies or custom designed bodies like tapered, curved or undulating designs. The balloon bodies may be cylindrical, or have alternative diametrical shapes, like ovals, lobed configurations, or more complex geometries.

Single or multiple chambers may be present on any of the balloon assemblies of the present invention.

A single chamber embodiment is shown in FIGS. 1A, 1B and 1C.

Multiple chambers may be configured in a concentric manner, as is illustrated for a two-chamber configuration in FIG. 5A, where a first chamber 221 surrounds a guidewire lumen 340, and is itself surrounded by a chamber 222, where chamber 222 may be a porous infusion chamber.

Many alternative embodiments are possible, as illustrated with examples for a spiral chamber 223 around a central inflation chamber 224 in FIG. 5B, where the spiral chamber 223 may be a porous infusion chamber, and for a series of parallel chambers 225, 226 and 227 as illustrated in FIG. 5C.

In some embodiments, design features may be added to a compliant balloon to minimize dimensional changes to the pores as a consequence of inflating the balloon. Specifically, if reinforcement features are incorporated in the balloon in conjunction with the pores, these reinforcements may protect the pores from dimensional changes, while still allowing the balloon to expand elastically.

For example, as shown for an exemplary embodiment of an uninflated compliant balloon chamber wall in FIG. 6A, can have one or more reinforcement features, such as raised protrusions 240 may be incorporated in the chamber wall 250 during a blow molding manufacturing process. The pores 210 of the balloon may be laser-drilled into the protrusions 240.

As shown after inflation, as shown in FIG. 6B, in this embodiment the elastic expansion of the chamber wall 250 may be mostly limited to the thin section of the wall 250, which may actually stretch to become thinner, while longitudinal stretch may position the pores 210 further apart. The thicker raised protrusions 240 may protect the pores 210 from significant dimensional changes during the expansion process, allowing better control of the release of the beneficial agent, such as the sclerotherapeutic agent.

In at least one embodiment, all of the pores 210 may be surrounded by or formed within protrusions 240. Alternatively, however, only some of the pores 210 may be surrounded by or formed within protrusions 240. Similarly, protrusions 240 can span the entire length and/or circumference of the balloon. In alternative embodiments, the protrusions 240 span only part of the length and/or part of the circumference of the balloon.

Furthermore, the protrusions 240 can form any number of patterns on the chamber wall 250. For instance, the protrusions 240 can be positioned on the chamber wall 250 in a manner that forms one or more rings about the circumference of the balloon. Additionally or alternatively, such rings may be concentric with the center axis of the balloon or may be oriented at a non-perpendicular angle relative to the center axis of the balloon. Further embodiments, the protrusions 240 can form spiral, linear, and any number of other suitable patterns on the surface of the chamber wall 250.

The protrusion 240 also may have any number of suitable configurations, which may vary from one embodiment to the next. In some embodiments, the protrusion 240 may entirely surround the pore 210. Alternatively, the protrusion 240 may only partially surround the pore 210. Hence, the pore 210 may exhibit different amount of expansion and deformation near the reinforced areas (i.e., near the protrusion(s) 240) and near unreinforced areas.

In one example, the protrusions 240 may be shaped as raised, longitudinal ridges, which partially surround corresponding pores 210 (e.g., the pores may be drilled in the ridges to form such balloon). Such longitudinal ridges may reduce or prevent the longitudinal expansion of the balloon chamber without inhibiting diametrical growth, thereby reducing or preventing the pores from moving apart during inflation. Hence, the protrusions 240 may be independent or separate from each other or may be connected to one or more of the adjacent protrusions 240, thereby forming one or more ridges on the surface of the balloon. As noted above, such ridges may form any number of patterns on the chamber wall 250.

Furthermore, in some embodiments, the protrusions 240 may comprise the same material as the chamber wall 250. Alternatively, the protrusions 240 may comprise a different material. For instance, the protrusions 240 can comprise a metallic material (e.g., stainless steel, nitinol, etc.), which can be overmolded with a more flexible thermoplastic or elastomeric material that can form the chamber wall 250. As such, the protrusions 240 may be substantially rigid and not deformable during inflation of the balloon, while the chamber wall 250 may deform and/or expand during inflation of the balloon.

Moreover, in some instances, reinforcements about the pores 210 may not protrude above the chamber wall 250 (or may protrude insignificantly). For example, as noted above, the protrusions 240 may comprise a more rigid material than the chamber wall 250. Likewise, the reinforcements generally may comprise more rigid material, such as stainless, steel, nitinol, etc. Accordingly, the pore reinforcements may be overmolded within the chamber wall 250 (e.g., encapsulated therein), in a manner that the pore reinforcements do not protrude above the surface of the chamber wall 250. In any event, the pore reinforcements, whether provided by protruding sections that at least partially surround the pore 210 or by sections of material that is more rigid than the chamber wall 250, can reduce or eliminate deformation and/or expansion of the pores 210 during the inflation of the balloon.

The exemplary embodiments above are merely included as illustrations of a wide range of possibilities. Those with ordinary skills in the art of balloon catheter design will be able to easily envision additional possible embodiments.

### Experimental Results

The examples described below are merely provided to demonstrate the present invention. They are based on exemplary embodiments, and are not to be interpreted as a limitation of the scope of the present invention.

In-Vitro Testing.

An in-vitro experiment was performed to demonstrate the feasibility of delivering a test solution in a continuous distribution to a synthetic model of a varicose vein.

The balloon catheters used were modifications of the commercially available 120 mm×6 mm Fox Plus® balloon catheters from Abbott Labs. The balloons were modified by laser drilling an evenly distributed pattern of 40 pores with a 30 micron diameter over the working length of the balloon.

Poly-vinyl alcohol (PVA) models of varicose veins were used as a test system. The models had an undulating design with inner diameters alternating between 3 mm and 6 mm, mimicking the appearance of a varicose vein.

The test fluid delivered was an aqueous solution of commercially available green food dye.

The balloons were inflated inside the vein models with the dye solution for duration of 1-3 minutes, with an inflation pressure of up to about 1 atmosphere, resulting in fluid delivery rates between approximately 5 and 10 ml/minute.

Upon examination of the PVA vein models after removal of the balloons, a continuous uptake of green dye over the entire segment of the vessel corresponding to the working length of the balloon was observed.

In-Vivo Testing

An animal study in an adult goat model was performed to demonstrate the efficacy of the present invention.

The balloon catheters used were modifications of the commercially available 60 mm×8 mm Armada 35® balloon catheters from Abbott Labs. The balloons were modified by laser drilling an evenly distributed pattern of 20 pores with a 25 micron diameter over the working length of the balloon. The selected balloon design was different than that in the in-vivo study, based on the anatomical requirements of the animal model, and on the desire to reduce the rate of delivery of the sclerotherapeutic agent. As mentioned above, modifications to the device can be made by those with ordinary skills in the art to accommodate for specific requirements of the biological environment of use.

The sclerotherapeutic agent formulation delivered was 96% ethanol.

The veins treated were the saphenous and cephalic veins in the hind limbs, resp front limbs of the goats. The animals were anesthetized during the procedure to reduce stress and discomfort and to facilitate their handling. A total of 3 animals were used, resulting in 12 vessels treated.

A maximum 15 ml of alcohol was infused into each of the veins, using pressures of up to 8 atmosphere and durations up to 3 minutes. The limbs were bandaged under compression after retrieval of the balloons, to limit thrombus accumulation in the treated vessels. After 14 days the animals were sacrificed and the veins harvested.

Histological examination revealed that out of 12 treated veins, 11 were fully occluded at the time of harvest, demonstrating the efficacy of the present invention.

Various parameters of the procedure described herein can easily be adjusted and are well within the scope of those with ordinary skills in the art.

As described herein, embodiments of the present invention can, among other things, facilitate treatment of varicose veins. For instance, one embodiment of the present invention includes a method for tumescent anesthesia-free treatment of a body lumen having a lumen wall. The method can involve positioning an inflatable balloon assembly at a location in the body lumen to be treated, the inflatable balloon assembly having a porous balloon chamber and one or more pores in an outer wall. The method also can involve inflating the porous balloon assembly into an inflated configuration in a manner that the outer wall of the inflatable balloon assembly abuts the lumen wall. Furthermore, the method can involve forcing a sclerotherapeutic agent to exit the one or more pores of the inflatable balloon assembly and to contact the lumen wall. In addition, the method can involve retaining the sclerotherapeutic agent between the lumen wall and the outer wall of the inflatable balloon assembly when the inflatable balloon assembly is in the inflated configuration.

Another embodiment can include a method for tumescent anesthesia-free treatment of a body lumen having a lumen wall. Such method can involve positioning an inflatable balloon assembly at a location in the body lumen to be treated, the inflatable balloon assembly having multiple porous balloon chambers folded around a guidewire. Moreover, the method can include unfolding and expanding the multiple porous balloon chambers of the inflatable balloon assembly into an unfolded configuration in a manner that outer walls of the multiple porous balloon chambers abut the lumen wall. Still further, the method can include forcing a sclerotherapeutic agent through one or more pores of the multiple porous balloon chambers to contact the lumen wall. The method also can include retaining the sclerotherapeutic agent between the lumen wall and the outer walls of the multiple porous balloon chambers when the multiple porous balloon chambers are in the unfolded configuration.

Another embodiment may include a device for administering a beneficial agent into a body lumen. As noted above, such beneficial agent may be a sclerotherapeutic agent. The device can include a balloon assembly having a compliant balloon chamber with a chamber wall and at least one pore in the chamber wall, the at least one pore being located at a reinforcement in the chamber wall.

It should be understood that the embodiments in this disclosure represent only a small fraction of the available options to practice the invention. In other words, the described embodiments are to be considered in all respects only as illustrative and not restrictive. Because of the broad applicability of the invention in the field sclerotherapy of body lumens, those with ordinary skills in the art will be able to design many additional embodiments, which will be within the scope of the invention. Accordingly, the present invention may be embodied in other specific forms without departing from its spirit or essential characteristics. The scope of the invention is, therefore, indicated by the appended claims rather than by the foregoing description. All changes that come within the meaning and range of equivalency of the claims are to be embraced within their scope.

What is claimed is:

1. A method for tumescent anesthesia-free treatment of a body lumen having a lumen wall, the method comprising:
positioning an inflatable balloon assembly at a location in the body lumen to be treated, the inflatable balloon assembly having a porous balloon chamber and one or more pores in an outer wall;
inflating the porous balloon assembly into an inflated configuration in a manner that the outer wall of the inflatable balloon assembly abuts the lumen wall;
forcing a sclerotherapeutic agent to exit the one or more pores of the inflatable balloon assembly and to contact the lumen wall; and
retaining the sclerotherapeutic agent between the lumen wall and the outer wall of the inflatable balloon assembly when the inflatable balloon assembly is in the inflated configuration.

2. The method of claim 1, further comprising deflating the inflatable balloon assembly, and retrieving the balloon assembly from the body lumen.

3. The method of claim 1, wherein the inflatable balloon assembly comprises not more than one porous balloon chamber.

4. The method of claim 1, wherein the inflatable balloon assembly comprises multiple porous balloon chamber.

5. The method of claim 1, wherein the inflatable balloon assembly comprises at least one non-porous balloon chamber.

6. The method of claim 1, wherein inflating the porous balloon assembly into an inflated configuration comprises inflating the porous balloon assembly with at least one inflation material.

7. The method of claim 6, wherein the at least one inflation material is the sclerotherapeutic agent.

8. The method of claim 1, wherein the body lumen is a vein.

9. The method of claim 1, wherein the porous balloon chamber is compliant.

10. The method of claim 1, wherein the porous balloon chamber is noncompliant.

11. A method for tumescent anesthesia-free treatment of a body lumen having a lumen wall, the method comprising:
positioning an inflatable balloon assembly at a location in the body lumen to be treated, the inflatable balloon assembly having multiple porous balloon chambers folded around a guidewire;
unfolding and expanding the multiple porous balloon chambers of the inflatable balloon assembly into an unfolded configuration in a manner that outer walls of the multiple porous balloon chambers abut the lumen wall;
forcing a sclerotherapeutic agent through one or more pores of the multiple porous balloon chambers to contact the lumen wall; and
retaining the sclerotherapeutic agent between the lumen wall and the outer walls of the multiple porous balloon chambers when the multiple porous balloon chambers are in the unfolded configuration.

12. The method of claim 11, further comprising folding multiple porous balloon chambers of the inflatable balloon assembly.

13. The method of claim 12, further comprising retrieving the inflatable balloon assembly from the body lumen.

14. The method of claim 11, wherein the inflatable balloon assembly comprises at least one non-porous balloon chamber.

15. The method of claim 11, wherein unfolding and expanding the multiple porous balloon chambers of the inflatable balloon assembly comprises inflating the multiple porous balloon chambers of the inflatable balloon assembly.

16. The method of claim 15, wherein inflating the multiple porous balloon chambers comprises inflating the multiple porous balloon chambers with at least one inflation material.

17. The method of claim 16, wherein the at least one inflation material is the sclerotherapeutic agent.

18. The method of claim 11, wherein the body lumen is a vein.

19. A device for administering a beneficial agent into a body lumen, the device comprising:
a balloon assembly having a compliant balloon chamber with a chamber wall; and
at least one pore in the chamber wall, the at least one pore being located at a reinforcement in the chamber wall.
